(19) United States
(12) Patent Application Publication
(10) Pub. No.: US 2022/0142819 A1
(43) Pub. Date: May 12, 2022

(54) SWIMMING EARPLUG

(71) Applicant: Shenzhen Taipuda Technology Co., Ltd., Shenzhen City (CN)

(72) Inventor: Dehua Guo, Shenzhen City (CN)

(21) Appl. No.: 17/403,894

(22) Filed: Aug. 17, 2021

(30) Foreign Application Priority Data
Nov. 9, 2020 (CN) 202022578602.9

(51) Int. Cl. A61F 11/12; A63B 33/00

(52) U.S. Cl. CPC A61F 11/12; A63B 33/00; A61F 2230/0093; A61F 2230/0023; A61F 2230/0065; A61F 2210/0076

(57) ABSTRACT

The present disclosure discloses a swimming earplug, which comprises an ear cap and a bracket, wherein the ear cap is a soft rubber ear cap, an ear cap through hole is provided at the center of the ear cap, the ear cap through hole penetrates the front and rear ends of the ear cap, an insertion post is formed at the front end of the bracket, the insertion post is inserted into the ear cap through hole and snugly fitted with the ear cap through hole, a bracket, through hole is provided in the bracket, the front end of the bracket through hole extends to a front end face of the insertion post such that the bracket through hole and the ear cap through hole communicate with each other, a rear end opening of the bracket through hole is covered with a waterproof and breathable membrane, and the waterproof and breathable membrane is fixedly connected to the bracket. Through the present disclosure, it is possible to effectively prevent water from entering an ear, the wearing comfort is better, and outside sounds can be heard clearly.

### TECHNICAL FIELD

The present disclosure relates to an earplug, and in particular to a swimming earplug.

### BACKGROUND ART

In the prior art, earplugs may be used in a variety of occasions, including noisy environments, bedroom environments, swimming occasions, etc. For people who like swimming, in order to prevent water from entering their ears, some swimmers will put earplugs in their ears. These earplugs are similar to anti-noise earplugs, which block ear canals in a manner of rebounding. Although this kind of earplug can play a waterproof role, its comfort during use is not good, and because of the inability to hear outside sounds, the swimmers cannot perceive instructions, sound signals, etc., transmitted by others in time. Most earplugs have the defects of being difficult to wear and easy to fall off and cannot meet the needs, of users and the market.

### SUMMARY

The technical problem to be solved by the present disclosure is to provide a swimming earplug that can be matched with multiple parts to facilitate wearing and improve stability, effectively prevent water from entering the ear, have better wearing comfort and make it possible to hear outside sounds, in view of the deficiencies of the prior art.

In order to solve the above technical problem, the technical solution used by the present disclosure is as follows.

A swimming earplug comprises an ear cap and a bracket, wherein the ear cap is a soft rubber ear cap, an ear cap through hole is provided at the center of the ear cap, the ear cap through hole penetrates the front and rear ends of the ear cap, an insertion post is formed at the front end of the bracket, the insertion post is inserted into the ear cap through hole and snugly fitted with the ear cap through hole, a bracket through hole is provided in the bracket, the front end of the bracket through hole extends to a front end face of the insertion post such that the bracket through hole and the ear cap through hole communicate with each other, a rear end opening of the bracket through hole is covered with a waterproof and breathable membrane, and the waterproof and breathable membrane is fixedly connected to the bracket.

Preferably, two snap rings protruding outward are formed on the side of the insertion post.

Preferably, the snap ring at the front end of the insertion post is a tapered snap ring.

Preferably, a step groove is provided at the edge of the rear end opening of the bracket through hole, and the waterproof and breathable membrane snaps in the step groove.

Preferably, both the step groove and the waterproof and breathable membrane are triangular.

Preferably, the swimming earplug comprises an ear brace, wherein an end of the ear brace is formed with a collar, the bracket is provided, with an annular slot, the collar is sleeved on the bracket, and the collar snaps in the annular slot.

Preferably, the inner side of the collar is provided with a positioning opening, a positioning block is formed in the annular slot, and the positioning block snaps in the positioning opening.

Preferably, the width of the annular slot is gradually reduced from the outside to the inside such that the cross section of the annular slot is trapezoidal, a wedge-shaped inner ring portion is formed on the inner side of the collar, and the inner ring portion is snugly fitted with the annular slot.

Preferably, the swimming earplug further comprises an ear brace, wherein the ear brace is provided with a main sleeve and a tail sleeve which communicate with each other, the bracket is provided with a main post and a tail post, the bracket through hole penetrates the main post and the tail post, the main sleeve is wrapped on the main post, and the tail sleeve is wrapped on the tail post.

Preferably, the outer wall of the main post is concave in the middle, and the inner wall of the main sleeve and the outer wall of the main post are attached to each other.

Preferably, the ear cap has a single-layer umbrella shape or a multi-layer umbrella shape.

Preferably, the waterproof and breathable membrane is made of metal, cloth, plastic, carbon fiber or composite material.

Preferably, a number of micropores are distributed on the waterproof and breathable membrane.

In the swimming earplug disclosed in the present disclosure, the soft rubber ear cap can fit well with the human ear canal, while reducing the pressure applied to the ear canal, and the wearing comfort is better. There is also a bracket through hole provided in the bracket. The bracket through hole and the ear cap through hole are kept in communication with each other. With the above two through holes, a user can clearly hear outside sounds. In order to prevent water from entering the ear through the above through holes, in the present disclosure, it is preferable to fix a waterproof and breathable membrane at an rear end opening of the bracket through hole. The waterproof and breathable membrane not only has a waterproof effect, but also can realize sound conduction under its breathability effect, so that the user can hear the outside sounds at any time, which satisfies the user's needs better.

### BRIEF DESCRIPTION OF THE DRAWINGS

FIG. 1 is a first three-dimensional view of a swimming earplug of the present disclosure;

FIG. 2 is a second three-dimensional view of a swimming earplug of the present disclosure;

FIG. 3 is a first exploded view of a swimming earplug of the present disclosure;

FIG. 4 is a second exploded view of a swimming earplug, of the present disclosure;

FIG. 5 is a three-dimensional view of another embodiment of a swimming earplug of the present disclosure; and

FIG. 6 is an exploded view of another embodiment of a swimming earplug of the present disclosure.

### DETAILED DESCRIPTION OF EMBODIMENTS

The following describes the present disclosure in more detail with reference to the drawings and embodiments.

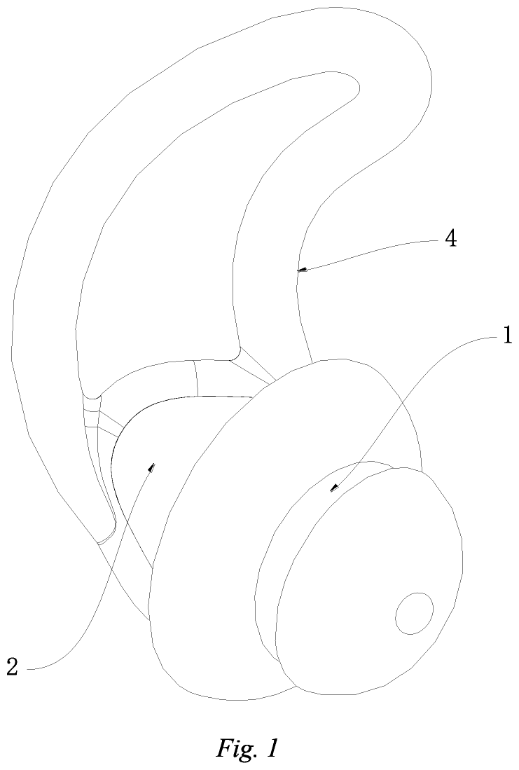

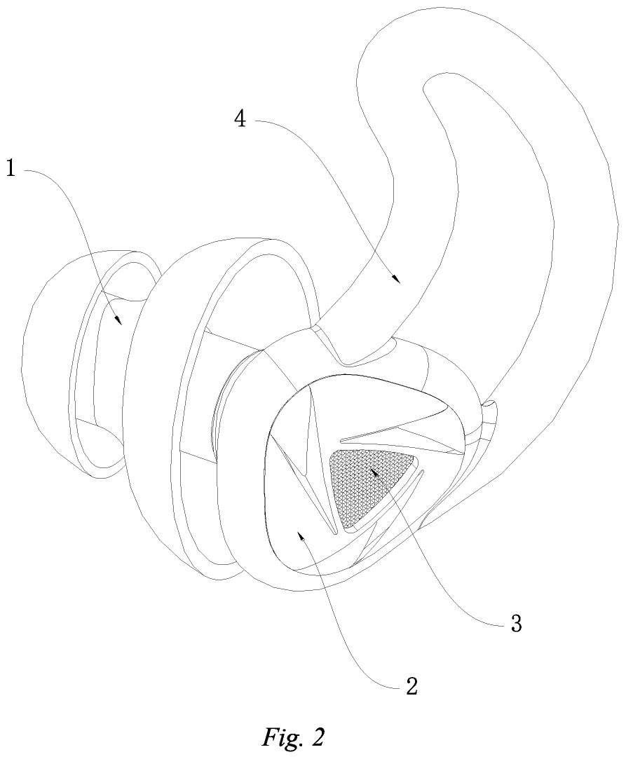

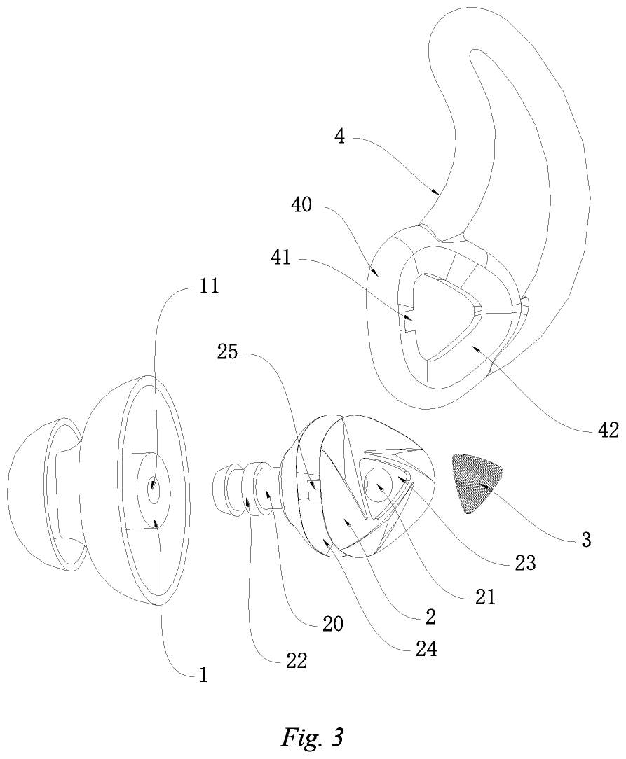

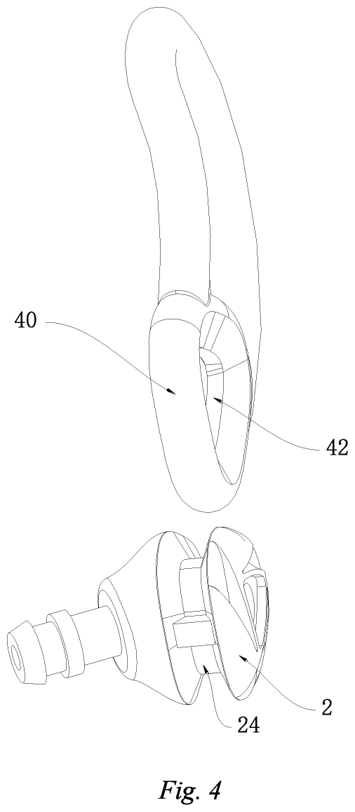

In a first embodiment, the present disclosure discloses a swimming earplug, which is shown in conjunction with FIG. 1 to FIG. 4, and comprises an ear cap 1 and a bracket 2. The ear cap 1 is a soft rubber ear cap, an ear cap through hole 11 is provided at the center of the ear cap 1, and the ear cap through hole 11 penetrates the front and rear ends of the ear cap 1. An insertion post 20 is formed at the front end of the bracket 2, and the insertion post 20 is inserted into the ear cap through hole 11 and snugly fitted with the ear cap through hole. A bracket through hole 21 is provided in the bracket 2, and the front end of the bracket through hole 21 extends to a front end face of the insertion post 20, such that the bracket through hole 21 and the ear cap through hole 11 communicate with each other. A rear end opening of the bracket through hole 21 is covered with a waterproof and breathable membrane 3, and the waterproof and breathable membrane 3 is fixedly connected to the bracket 2.

In the above structure, the soft rubber ear cap can fit well with the human ear canal, while reducing the pressure applied to the ear canal, and the wearing comfort is better. There is also a bracket through hole 21 provided in the bracket 2. The bracket through hole 21 and the ear cap through hole 11 are kept in communication with each other. With the above two through holes, a user can clearly hear outside sounds. In order to prevent water from entering the ear through the above through holes, in the present disclosure, it is preferable to fix a waterproof and breathable membrane 3 at an rear end opening of the bracket through hole 21. The waterproof and breathable membrane 3 not only has a waterproof effect, but also can realize sound conduction under its breathability effect, so that the user can hear the outside sounds at any time, which satisfies the user's needs better.

In the first embodiment, two snap rings 22 protruding outward are formed on the side of the insertion post 20. With the snap rings 22, the insertion post 20 can be more snugly fitted with the ear cap 1.

In order to facilitate insertion of the insertion post 20, in the first embodiment, the snap ring 22 at the front end of the insertion post 20 is a tapered snap ring.

In order to accommodate and fix the waterproof and breathable membrane 3, in the first embodiment, a step groove 23 is provided at the edge of the rear end opening of the bracket through hole 21, and the waterproof and breathable membrane 3 snaps in the step groove 23.

Preferably, both the step groove 23 and the waterproof and breathable membrane 3 are triangular. In a practical application, the waterproof and breathable membrane 3 may be a metal mesh or a mesh cloth with dense holes.

In a practical application, the bracket 2 should also be provided in a generally triangular structure, which can better match with the human ear.

In order to prevent the swimming earplug from falling off, the first, embodiment comprises an ear brace 4, and an end of the ear brace 4 is formed with a collar 40. The bracket 2 is provided with an annular slot 24, the collar 40 is sleeved on the bracket 2, and the collar 40 snaps in the annular slot 24.

In a practical application, the ear brace 4 has a shape that protrudes outward and resembles a shark fin, and the ear brace 4 of this shape can better engage with the human ear, thereby playing a role of preventing the swimming earplug from falling off.

In addition, common earplugs in the prior art are generally forcibly inserted into the ear canal by an external force. This insertion method will make the earplugs difficult to put in and take out, unstable, and easy to fall off. In contrast, in the present disclosure, the ear brace 4 made of soft rubber is mounted on the bracket 2, and with the elastic support effect, of the ear brace 4, it is possible to engage with an auricle more stably and reliably, thus effectively avoiding the earplug body from falling out. In addition, in the present disclosure, a structure in which an ear cap and an ear hook are fixed on a bracket is adopted. This combination makes the earplugs easy to wear and take out, which better meets the application requirements.

Further, the inner side of the collar 40 is provided with a positioning opening 41, a positioning block 25 is formed in the annular slot 24, and the positioning block 25 snaps in the positioning opening 41.

The above structure can make the collar 40 better engage with the bracket 2 and at the same time can prevent the collar 40 from rotating and shifting.

In order to facilitate the disassembly and assembly of the ear brace 4, in the first embodiment, the width of the annular slot 24 is gradually reduced from the outside to the inside, such that the cross, section of the annular slot 24 is trapezoidal. A wedge-shaped inner ring portion 42 is formed on the inner side of the collar 40, and the inner ring portion 42 is snugly fitted with the annular slot 24.

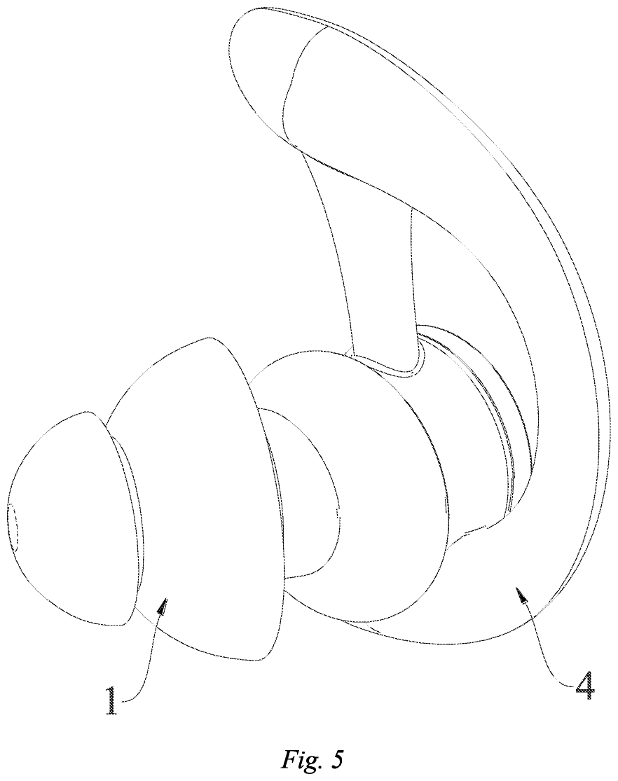

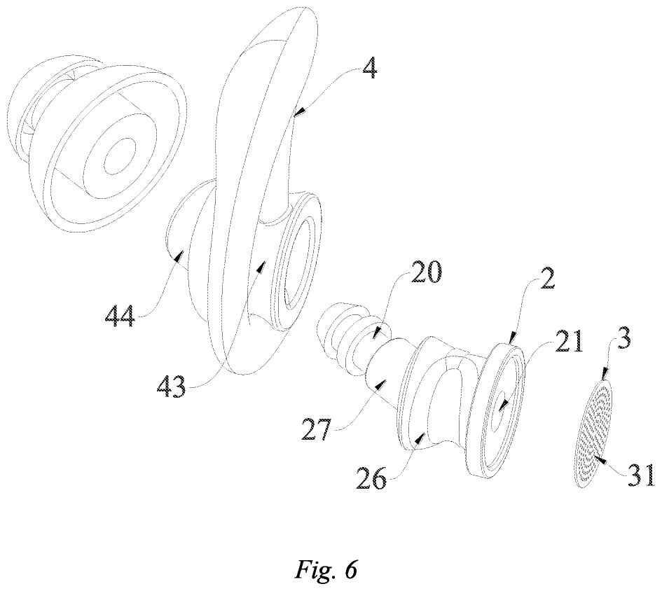

FIG. 5 and FIG. 6 together show the structure of a swimming earplug of a second embodiment. As with the first embodiment, as shown in FIG. 5, the ear cap 1 is a double-layer umbrella-shaped ear cap made of soft rubber. In other embodiments, the ear cap can also be designed into a single-layer umbrella-shaped ear cap or a three-layer umbrella-shaped ear cap as required. Only the differences between the swimming earplug structures of the second embodiment and the first embodiment will be described below.

As shown in FIG. 5, the ear brace 4 is made into a “D” shape. The ear brace 4 is integrally made. In implementation, the ear brace 4 can be designed to match the shape of the user's auricle, and the shape of the ear brace 4 is not limited herein.

As shown in FIG. 6, the waterproof and breathable membrane 3 is made into a disc shape and has a number of micropores 31 distributed thereon. Due to the existence of the micropores 31, water cannot, pass through the micropores 31 due to surface tension, while sound can pass through the micropores 31 but the transmitted sound is attenuated. In implementation, the waterproof and breathable membrane 3 can be made of metal, cloth, plastic, carbon fiber or composite material.

As shown in FIG. 6, the ear brace 4 is provided with a main sleeve 43 and a tail sleeve 44 which communicate with each other. The bracket 2 is provided with a main post 26 and a tail post 27, and the bracket through hole 21 penetrates the main post 26 and the tail post 27. The main sleeve 43 is wrapped on the main post 26, while the tail sleeve 44 is wrapped on the tail post 27, which enhances the wrapping and improves the use experience. The outer wall of the main post 26 is concave in the middle, and the inner wall of the main sleeve 43 and the outer wall of the main post 26 are attached to each other, such that the main sleeve 43 of the ear brace 4 is stuck by the main post 26, so the ear brace 4 is not easily separated from the bracket 2.

The above embodiments are merely preferred embodiments of the present disclosure but not intended to limit the present disclosure, and any modifications, equivalent replacements, improvements, etc. made within the spirit and principle of the present disclosure should be included within the scope of protection of the present disclosure.

What is claimed is:

1. A swimming earplug, comprising an ear cap (1) and a bracket (2), wherein the ear cap (1) is a soft rubber ear cap, an ear cap through hole (11) is provided at the center of the ear cap (1), the ear cap through hole (11) penetrates the front and rear ends of the ear cap (1), an insertion post (20) is formed at the front end of the bracket (2), the insertion post (20) is inserted into the ear cap through hole (11) and snugly fitted with the ear cap through hole, a bracket through hole (21) is provided in the bracket (2), the front end of the bracket through hole (21) extends to a front end face of the insertion post (20) such that the bracket through hole (21) and the ear cap through hole (11) communicate with each other, a rear end opening of the bracket through hole (21) is covered with a waterproof and breathable membrane (3), and the waterproof and breathable membrane (3) is fixedly connected to the bracket (2).

2. The swimming earplug of claim 1, wherein two snap rings (22) protruding outward are formed on the side of the insertion post (20), and the snap ring 22 at the front end of the insertion post 20 is a tapered snap ring.

3. The swimming earplug of claim 1, wherein a step groove (23) is provided at the edge of the rear end opening of the bracket through hole (21), and the waterproof and breathable membrane (3) snaps in the step groove (23).

4. The swimming earplug of claim 3, wherein both the step groove (23) and the waterproof and breathable membrane (3) are triangular.

5. The swimming earplug of claim 1, comprising an ear brace (4), wherein an end of the ear brace (4) is formed with a collar (40), the bracket (2) is provided with an annular slot (24), the collar (40) is sleeved on the bracket (2), and the collar (40) snaps in the annular slot (24).

6. The swimming earplug of claim 5, wherein the inner side of the collar (40) is provided with a positioning opening (41), a positioning block (25) is formed in the annular slot (24), and the positioning block (25) snaps in the positioning opening (41).

7. The swimming earplug of claim 5, wherein the width of the annular slot (24) is gradually reduced from the outside to the inside such that the cross section of the annular slot (24) is trapezoidal, a wedge-shaped inner ring portion (42) is formed on the inner side of the collar (40), and the inner ring portion (42) is snugly fitted with the annular slot (24).

8-12. (canceled)
